(19) United States
(12) Patent Application Publication
(10) Pub. No.: US 2004/0141936 A1
(43) Pub. Date: Jul. 22, 2004

(54) Use of sodium polystyrene sulfonate for tightening skin

(76) Inventors: Rainer Kropke, Schenefeld (DE); Jens Nielsen, Henstedt-Ulzburg (DE)

Correspondence Address: Greenblum & Bernstein, 1950 Roland Clarke Place, Reston, VA, 20191, US

(21) Appl. No.: 10/478,294

(22) PCT Filed: May 31, 2002
(86) PCT No.: PCT/EP02/05950

(30) Foreign Application Priority Data
Jun. 2, 2001 (DE) 10127087.9

(51) Int. Cl. A61K 7/06; A61K 7/11

(52) U.S. Cl. 424/70.11

(57) ABSTRACT

Use of sodium polystyrenesulfonate for producing cosmetic or dermatological preparations for tightening and/or firming the skin.

The present invention relates to cosmetic or dermatological dermatological preparations comprising active ingredients for the care and protection of the skin, in particular sensitive skin, and, very particularly, skin aging or aged by intrinsic and/or extrinsic factors, and the use of such active ingredients and combinations of such active ingredients in the field of cosmetic and dermatological skincare.

Cosmetic skincare primarily means the strengthening or rebuilding of the skin's natural function as a barrier against environmental influences (e.g. dirt, chemicals, microorganisms) and against the loss of endogenous substances (e.g. water, natural fats, electrolytes).

If this function is impaired, increased resorption of toxic or allergenic substances or attack by microorganisms may result, leading to toxic or allergic skin reactions.

Another aim of skincare is to compensate for the loss by the skin of lipids and water caused by daily washing. This is particularly important when the natural regeneration ability is insufficient. Furthermore, skincare products should protect against environmental influences, in particular against sun and wind, and delay skin ageing.

Chronological skin ageing is caused, for example, by endogenous genetically determined factors. The following structural damage and functional disorders, which can also fall under the term “senile xerosis”, result, for example, in the epidermis and dermis as a result of ageing:

a) dryness, roughness and the formation of dryness wrinkles,

b) itching and

c) reduced refatting by sebaceous glands (e.g. after washing).

Exogenous factors, such as UV light and chemical noxae, can have a cumulative effect and, for example, accelerate or supplement the endogenous ageing processes. In the epidermis and dermis, for example, the following structural damage and functional disorders appear in the skin as a result of exogenous factors; these go beyond the extent and quality of the damage in the case of chronological ageing:

d) visible vascular dilation (telangiectases, couperosis);

e) flaccidity and formation of wrinkles;

f) local hyper-, hypo- and abnormal pigmentation (e.g. age spots) and

g) increased susceptibility to mechanical stress (e.g. cracking).

The present invention relates in particular to products for the care of skin aged naturally, and to the phenomena listed under a), e) and g).

Products for the care of flaccid, in particular aged, skin are known per se. They comprise, for example, retinoids (vitamin A acid and/or derivatives thereof) or vitamin A and/or derivatives thereof. Their action on structural damage is, however, limited in terms of extent. Furthermore, in product development there are considerable difficulties in stabilizing the active ingredients to an adequate extent against oxidative decay. The use of products comprising vitamin A acid, moreover, often causes severe erythematous skin irritation. Retinoids can therefore only be used in low concentrations.

Flaccid skin is often also associated with an accompanying phenomenon of excess weight and/or cellulite, which is often associated therewith.

The body awareness of consumers has increased significantly in recent years. In this connection, as well as cleansing and care applications, measures are increasingly being taken to improve the silhouette of the body. Cellulite—a widespread phenomenon—assumes a central position. The visible appearance of cellulite is based on an increase of fatty bodies in the subcutis (subcutaneous fatty tissue), a weakening of the connective tissue and a reduction in the through-flow ratio in the blood stream and lymphatic tract. The cause is therefore a partly position-dependent weakening of the connective tissue with the simultaneous occurrence of enlarged fatty cell chambers as a result of excess weight, an unbalanced diet and lack of exercise. The formation of cellulite can also be attributed to increased permeability of the capillary walls, which permits penetration of water into the connective tissue.

In addition, there may be a localized testosterone deficiency at the areas of skin affected. In any case, cellulite is a phenomenon which is almost never observed in men.

The object of the present invention was therefore to find ways to avoid the disadvantages of the prior art. In particular, it was an object of the present invention to provide preparations which can effect advantageous tightening of flaccid skin.

Surprisingly, it has been found that the use of sodium polystyrenesulfonate for producing cosmetic or dermatological preparations for tightening the skin overcomes the disadvantages of the prior art.

Sodium polystyrenesulfonate is sold, for example, under the trade name Flexan®. It is a high molecular weight polystyrenesulfonate with anionic character and is available as a 30% strength aqueous solution; it corresponds to the formula below; its CTFA name is “Sodium Polystyrene Sulfonate”. Due to its ionic character, it is readily conductive and, in low concentrations, prevents the charging of hair in hair cosmetic preparations.

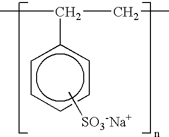

According to the invention, the use of sodium polystyrenesulfonate for producing cosmetic or dermatological preparations for the cosmetic and/or dermatological treatment and/or prophylaxes of cellulite is particularly advantageous.

The amount of sodium polystyrenesulfonate the preparations is preferably 0.001 to 30% by weight, particularly preferably 0.05-20% by weight, in particular 1-10% by weight, based on the total weight of the preparation.

According to the invention, it is particularly highly advantageous to use the active ingredient or cosmetic or topical dermatological preparations used according to the invention with an effective content of active ingredient, used according to the invention, for the cosmetic or dermatological treatment or prophylaxis of undesired skin conditions.

According to the invention, customary antioxidants can be used to the preparations which comprise the active ingredient combinations according to the invention.

The antioxidants are advantageously chosen from the group consisting of amino acids (e.g. glycine, histidine, tyrosine, tryptophan, proline) and derivatives thereof, imidazoles (e.g. urocanic acid) and derivatives thereof, peptides, such as D,L-carnosine, D-camosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to pmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechins, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations having an effective content of active ingredient used according to the invention is carried out in the usual manner, mainly by applying the active ingredient used according to the invention or the cosmetic or topical dermatological preparations having an effective content of active ingredient used according to the invention to the affected areas of skin.

Emulsions according to the invention for the purposes of the present invention, e.g. in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without customary auxiliaries and additives. Examples thereof include bodying agents, fillers, perfume, dyes, emulsifiers, additional active ingredients, such as vitamins or proteins, light protection agents, stabilizers, insect repellents, alcohol, water, salts, and antimicrobially, proteolytically or keratolytically active substances etc.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

In this connection, it is likewise advantageous to add the active ingredient used according to the invention as an additive to preparations which already comprise other active ingredients for other purposes.

Accordingly, for the purposes of the present invention, cosmetic or topical dermatological compositions can, depending on their formulation, be used, for example, as skin protection cream, cleansing milk, sunscreen lotion, nourishing cream, day or night cream, etc. In some instances it is possible and advantageous to use the compositions according to the invention as bases for pharmaceutical formulations.

It is, however, also advantageous for the purposes of the present invention to provide cosmetic and dermatological preparations whose main purpose is not protection against sunlight, but which nevertheless have a content of UV protection substances. Thus, for example, UV-A and/or UV-B filter substances are usually incorporated into day creams.

Preparations according to the invention can advantageously comprise substances which absorb UV radiation in the UVB region, the total amount of filter substances being, for example, 0.1% by weight to 30% by weight, preferably 0.5 to 10% by weight, in particular 1 to 6% by weight, based on the total weight of the preparations.

The UVB filters can be oil-soluble or water-soluble. Examples of oil-soluble substances are:

3-benzylidenecamphor and derivatives thereof, e.g. 3-(4-methylbenzylidene)camphor,

4-aminobenzoic acid derivatives, preferably 2-ethylhexyl 4-(dimethylamino)benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy4′-methylbenzophenone, 2,2′-dihydroxy-4-methoxybenzophenone;

esters of benzalmalonic acid, preferably di(2-ethylhexyl) 4-methoxybenzalmalonate;

2,4,6-trianilino(p-carbo-2′-ethyl-1′-hexyloxy)-1,3,5-triazine.

Advantageous water-soluble substances are:

2-phenylbenzimidazole-5-sulphonic acid and salts thereof, e.g. sodium, potassium or triethanolammonium salts,

sulfonic acid derivatives of benzophenones, preferably 2-hydroxy-4-methoxybenzophenone-5-sulphonic acid and its salts;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and its salts.

The list of said UVB filters which can be used according to the invention is of course not intended to be limiting.

The invention also provides the combination of a UVA filter according to the invention with a UVB filter or a cosmetic or dermatological preparation according to the invention which also comprises a UVB filter.

It can also be advantageous to use UVA filters which are customarily p resent in cosmetic and/or dermatological preparations in preparations according to the invention. Such filter substances are preferably derivatives of dibenzoylmethane, in particular 1-(4′-tertbutylphenyl)-3-(4′-methoxyphenyl)propane-1,3-dione and 1-phenyl-3-(4′-isopropylphenyl)-propane-1,3-dione. Preparations which comprise these combinations are also provided by the invention. It is possible to use the same amounts of UVA filter substances which have been given for UVB filter substances.

Cosmetic and/or dermatological preparations for the purpose of the present invention can also comprise inorganic pigments which are customarily used in cosmetics for protecting the skin against UV rays. These are oxides of titanium, zinc, iron, zirconium, silicon, manganese, aluminum, cerium and mixtures thereof, and modifications in which the oxides are the active agents. Particular preference is given to pigments based on titanium dioxide. It is possible to use the amounts given for the above combinations.

The cosmetic and dermatological preparations according to the invention can comprise cosmetic active ingredients, auxiliaries and/or additives as are customarily used in such preparations, e.g. antioxidizing agents, preservatives, bactericides, perfumes, antifoams, dyes, pigments which have a coloring action, thickeners, surface-active substances, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

If the cosmetic or dermatological preparation for the purposes of the present invention is a solution or emulsion or dispersion, solvents which may be used are:

water or aqueous solutions

oils, such as triglycerides of capric or caprylic acid, but preferably castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water can be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24 carbon atoms, in particular 12-18 carbon atoms. The fatty acid triglycerides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous mixtures are those of C₁₂₋₁₅-alkyl benzoate and 2-ethylhexyl isostearate, those of C₁₂₋₁₅-alkyl benzoate and isotridecyl isononanoate, and those of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

Advantageously, the oil phase can also have a content of cyclic or linear silicone oils, or consist entirely of such oils, although it is preferred to use an additional content of other oil phase components apart from the silicone oil or the silicone oils.

Cyclomethicone (octamethylcyclotetrasiloxane) is advantageously used as the silicone oil to be used according to the invention. However, other silicone oils can also be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

The aqueous phase of the preparations according to the invention optionally advantageously comprises

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol and, in particular, one or more thickeners which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of Carbopols, for example Carbopol grades 980, 981, 1382, 2984, 5984, in each case individually or in combination.

Customary bases which are suitable for use as cosmetic sticks for the purposes of the present invention are liquid oils (e.g. paraffin oils, castor oil, isopropyl myristate), semisolid constituents (e.g. vaseline, lanolin), solid constituents (e.g. beeswax, ceresine and microcrystalline waxes and ozokerite) and high-melting waxes (e.g. carnauba wax, candelilla wax)

Suitable propellants for cosmetic and/or dermatological O/W, emulsions which can be sprayed from aerosol containers for the purposes of the present invention are the customary known readily volatile, liquefied propellants, for example hydrocarbons (propane, butane, isobutane), which can be used alone or in a mixture with one another. Compressed air is also used advantageously.

The person skilled in the art is of course aware that there are propellants which are nontoxic per se and are in principle suitable for realizing the present invention in the form of aerosol preparations, but which must nevertheless be avoided because of their unacceptable impact on the environment or other accompanying circumstances, in particular fluorinated hydrocarbons and chlorofluorocarbons (CFCs).

For the purposes of the present invention, cosmetic preparations can also be in the form of gels which, in addition to an effective content of the active ingredient according to the invention and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

### EXAMPLES 1-10

### O/W Creams

| | Example number | | | | | | | | | |
|---|---|---|---|---|---|---|---|---|---|---|
| | 1 | 2 | 3 | 4 | 5 | 6 | 7 | 8 | 9 | 10 |
| Glyceryl stearate citrate | 2 | | | | | | | | | |
| Glyceryl stearate, self- | | 5 | | | | | | | | |
| emulsifying | | | | | | | | | | |
| PEG-40 stearate | | | 3 | | 0.7 | | | | | |
| PEG-100 stearate | | | | | | 1 | | | | |
| Polyglyceryl-3 methylglucose | | | | 2 | | | 3 | | | |
| distearate | | | | | | | | | | |
| Sorbitan stearate | | | | | | | 1 | | | |
| Polyethylene glycol(21) | | | | | | | | 2 | | |
| stearyl ether (Steareth-21) | | | | | | | | | | |
| Polyethylene glycol(2) stearyl | | | | | | | | 1 | | |
| ether (Steareth-2) | | | | | | | | | | |
| Cetearyl glucoside | | | | | | | | | 2 | |
| Stearic acid | | | | | | | | | | 2.5 |
| Myristyl myristate | 1 | | | | 1 | | | | 1 | |
| Behenyl alcohol | | | | | | | 1 | | | 2 |
| Stearyl alcohol | 2 | 1 | | | | | | | 5 | |
| Cetearyl alcohol | | | | | 2 | | | 2 | | 1 |
| Cetyl alcohol | 1 | | 2 | 2 | | 1 | 1 | | | |
| Hydrogenated coconut fatty | 2 | | | | | 1 | | | | 1 |
| glycerides (hydrogenated | | | | | | | | | | |
| cocoglycerides) | | | | | | | | | | |
| Shea butter | | 2 | | | | | 2 | | | |
| C12-15 alkyl benzoate | | 3 | 1 | 2 | 3 | | 2 | | 2 | |
| Butylene glycol dicaprylate/ | 1 | | | | | | | | | 2 |
| dicaprate | | | | | | | | | | |
| Caprylic/capric triglycerides | | 1 | 4 | 2 | | | 1 | | | |
| Hydrogenated polydecene | | | | | | | | | 1 | |
| Ethylhexyl coconut fatty acid | 3 | | | | | | | | | 2 |
| ester | | | | | | | | | | |
| Octyldodecanol | | | 0.5 | | | | | 1 | | |
| Mineral oil | | 1 | | | | | | 3 | | |
| Vaseline | 4 | | | | | 2 | | | | |
| Octamethyltetrasiloxane | | 1 | 3 | 6 | 2 | 1 | | 2 | | |
| (cyclomethicone) | | | | | | | | | | |
| Dimethylpolysiloxane | | | | | 1 | | | | | 1 |
| (dimethicone) | | | | | | | | | | |
| Dicaprylyl ether | 1 | 4 | | | | | | | | |
| Dicarprylyl carbonate | | | | | | | 2 | | | 4 |
| Polydecene | | | | | | 1 | | | 5 | |
| Ubiquinone (Q10) | | 0.1 | | | | 0.1 | | | 0.1 | |
| Tocopheryl acetate | 1 | | 0.5 | | | | 0.5 | | | |
| α-Glucosylrutin | | | | | 0.1 | | | | | |
| Biotin | | | | | | 0.1 | | 0.1 | | |
| Iminodisuccinate | 0.1 | 0.1 | | 0.2 | 0.3 | 0.3 | 0.5 | 0.1 | | 0.1 |
| Phenoxyethanol | 0.3 | | 0.3 | 0.2 | | 0.8 | 0.4 | 0.5 | | 0.3 |
| p-Hydroxybenzoic alkyl ester | 0.6 | 0.4 | 0.2 | 0.3 | 0.3 | 0.4 | | | 0.4 | 0.6 |
| (paraben) | | | | | | | | | | |
| Hexamidine diisethionate | | | | | 0.04 | 0.05 | | 0.1 | | |
| Diazolidinyl urea | 0.25 | | 0.1 | | | | 0.2 | | 0.1 | |
| 1,3-Dimethylol-5,5-dimethyl- | | 0.2 | | | | | | | | |
| hydantoin (DMDM | | | | | | | | | | |
| hydantoin) | | | | | | | | | | |
| Iodopropynyl butylcarbamate | 0.1 | | | 0.1 | 3 | | | 0.25 | | |
| Ethanol denatured | 1 | 2 | | 2 | | | 8 | | | 3 |
| 2-Ethylhexyl glycerol ether | | | | | | 3 | | | | |
| (octoxyglyceryl) | | | | | | | | | | |
| Xanthan gum | 0.6 | | 0.1 | | | 0.2 | 0.1 | | | |
| Polyacrylic acid (carbomer) | 0.05 | | | 0.3 | 0.1 | | | 0.1 | | |
| Polyacrylamide | | 0.2 | | | | | 0.2 | | | |
| Glycerol | 5 | 10 | 7.5 | 5 | 5 | 15 | 5 | 6 | 4 | 2 |
| Butylene glycol | 2 | 1 | | | 2 | | | | 2 | |
| Water- and/or oil-soluble | 0.05 | | | | | | | | | 0.1 |
| dyes | | | | | | | | | | |
| Fillers (distarch phosphate, | | | | 0.5 | | | | | 5 | 1 |
| SiO₂, talc, aluminum | | | | | | | | | | |
| stearate) | | | | | | | | | | |
| Sodium polystrenesulfonate | 3 | 2 | 4 | 2.5 | 10 | 12 | 3 | 2 | 4 | 2 |
| Proline | 1 | 0.3 | 0.1 | 0.1 | | 1 | 0.3 | 0.1 | | |
| Alanine | | 0.4 | 0.2 | | | | | | | |
| Arginine | | | | | 1 | 0.3 | 0.1 | | 0.4 | 0.2 |
| Caffeine | 0.15 | 0.4 | 0.2 | 0.1 | | 0.4 | 0.3 | 1 | 0.3 | 0.1 |
| Perfume | q.s. | q.s. | q.s. | q.s. | q.s. | q.s. | q.s. | q.s. | q.s. | q.s. |
| Water | ad 100 | ad 100 | ad 100 | ad 100 | ad 100 | ad 100 | ad 100 | ad 100 | ad 100 | ad 100 |

### EXAMPLE 11

### W/O Cream

| | |
|---|---|
| Polyglyceryl-3 diisostearate | 5.0 |
| Polyglyceryl-2 dipolyhydroxystearate | 2.5 |
| Cetearyl alcohol | 2 |
| Cetyl alcohol | 2 |
| C12-15 alkyl benzoate | 10 |
| Caprylic/capric triglycerides | 5 |
| Octyldodecanol | 7 |
| Octamethyltetrasiloxane (cyclomethicone) | 2 |
| Lactic acid | 5 |
| Aluminum chlorohydrate | 1 |
| β-Cyclodextrin | 0.5 |
| Distarch phosphate | 1 |
| Phenoxyethanol | 0.1 |
| p-Hydroxybenzoic alkylester (paraben) | 0.1 |
| Glycerol | 5 |
| Fillers (distarch phosphate, SiO₂, talc, | 0.2 |
| aluminum stearate) | |
| Sodium polystyrenesulfonate | 10 |
| Perfume | q.s. |
| Water | ad 100 |

### EXAMPLE 12

### Hydrodispersion/gel Cream

| | |
|---|---|
| Cetearyl alcohol | 1 |
| Shea butter | 1 |
| Caprylic/capric triglycerides | 2 |
| Octyldodecanol | 1 |
| Octamethyltetrasiloxane (cyclomethicone) | 4 |
| Dimethylpolysiloxane (dimethicone) | 1 |
| Polydecene | 2 |
| Ethylhexyl methoxycinnamate | 2 |
| Ubiquinone (Q10) | 0.1 |
| Aluminum chlorohydrate | 0.2 |
| β-Cyclodextrin | 0.5 |
| Distarch phosphate | 1 |
| Phenoxyethanol | 0.3 |
| p-Hydroxybenzoic alkyl ester (paraben) | 0.4 |
| Polyacrylic acid (carbomer) | 0.1 |
| Crosslinked alkyl acrylate (alkyl acrylates | 0.2 |
| crosspolymer) | |
| Sodium polystrenesulfonate | 5 |
| Glycerol | 5 |
| Perfume | q.s. |
| Water | ad 100 |

1. The use of sodium polystyrenesulfonate for producing cosmetic or dermatological preparations for tightening and/or firming the skin.

2. The use as claimed in claim 1 for the cosmetic and/or dermatological treatment and/or prophylaxes of cellulite.

3. The use as claimed in claim 1, characterized in that the amount of sodium polystyrenesulfonate the preparations is 0.001 to 30% by weight, particularly preferably 0.05-20% by weight, in particular 1-10% by weight, based on the total weight of the preparation.
